(19) United States
(12) Patent Application Publication
(10) Pub. No.: US 2025/0259729 A1
(43) Pub. Date: Aug. 14, 2025

(54) HUMAN-ARTIFICIAL INTELLIGENCE INTERACTION FOR PHYSICAL THERAPY MEASUREMENTS

(71) Applicants: Amit Gur, Zichron-Yaakov (IL); Omer Achrack, Jerusalem (IL); Yael Yankelevsky, Haifa (IL); Ron Alfia, Haifa (IL); Alexandra Manevitch, Jerusalem (IL); Riki Sheinin, Jerusalem (IL); Mohr Wenger, Gedera (IL); Uzi Sarel, Zichron-Yaakov (IL)

(72) Inventors: Amit Gur, Zichron-Yaakov (IL); Omer Achrack, Jerusalem (IL); Yael Yankelevsky, Haifa (IL); Ron Alfia, Haifa (IL); Alexandra Manevitch, Jerusalem (IL); Riki Sheinin, Jerusalem (IL); Mohr Wenger, Gedera (IL); Uzi Sarel, Zichron-Yaakov (IL)

(21) Appl. No.: 19/193,257

(22) Filed: Apr. 29, 2025

(51) Int. Cl. G16H 20/30; A61B 5/11; G16H 40/67

(52) U.S. Cl. CPC G16H 20/30; A61B 5/1128; G16H 40/67

(57) ABSTRACT

Systems and methods for automatically analyzing movements to identify anomalies and incorrect actions. An artificial intelligence-based digital system is provided that can monitor exercises and automatically analyze movements to identify both anomalies and incorrect actions. The system includes a mobile device application that can serve as a user interface for the patient, a camera for streaming video of the patient performing the exercise in real-time, and an artificial intelligence-based system to analyze the patient's movements while performing the exercise and provide real time guidance and feedback to the patient. The system includes a video dataset including people performing a set a supported exercises. In some examples, the video dataset can be used to adjust checks of the artificial intelligence system for metric accuracy. The system can be used for physical therapy treatment tracking, ensuring patients are accurately performing exercises.

### CROSS-REFERENCE TO RELATED APPLICATION

This application is related to concurrently filed U.S. Application titled “Human Activity Analysis Based on Skeleton and Deep Learning Transformer”, which is hereby incorporated by reference in its entirety.

### TECHNICAL FIELD

This disclosure relates generally to object recognition, and in particular object recognition for movement analysis.

### BACKGROUND

In Physical Therapy (PT) treatment, it is important to accurately identify when a patient performs an exercise incorrectly and provide corrective actions. Additionally, PT treatment plans are adjusted based on detected movement anomalies. The variety of exercises used by the Physical Therapy (PT) community is vast, ranging from 5,000 to 15,000 unique exercises. For each exercise, the number of potential incorrect movements or anomalies is also extensive and unpredictable. Furthermore, while performing their PT exercises, patients may also engage in many unrelated activities that are not part of the required exercise regimen. These factors present challenges to the development of automated systems for providing PT treatment.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, and not by way of limitation, in the figures of the accompanying drawings.

FIG. 1 illustrates a deep learning system, in accordance with various embodiments.

FIG. 2 illustrates an example overview of an exercise analysis system 200, in accordance with various embodiments.

FIG. 3 illustrates an example of a pipeline for implementation of the AI-based exercise analysis module, in accordance with various embodiments.

FIG. 4 is a flowchart showing a method 400 of exercise analysis, in accordance with various embodiments.

FIGS. 5A-5C illustrate examples of a user interface for an exercise analysis system, in accordance with various embodiments.

FIG. 6 is a block diagram of an example computing device, in accordance with various embodiments.

### DETAILED DESCRIPTION

### Overview

An artificial intelligence-based digital system is provided herein that can monitor PT exercises and automatically analyze movements to identify both anomalies and incorrect actions. The system includes a mobile device application that can serve as a user interface for the patient, a camera for streaming video of the patient performing the exercise in real-time, and an artificial intelligence-based system to analyze the patient's movements while performing the exercise and provide real time guidance and feedback to the patient. In some implementations, the system also includes a video dataset including people performing a set a supported exercises. In some examples, the video dataset can be used to adjust checks of the artificial intelligence system for metric accuracy.

In automated PT treatment tracking systems, and in particular in automated PT treatment tracking systems based on a single two-dimensional (2D) camera, measuring patient metrics across all possible human movements is very challenging. There are approximately 5,000 to 15,000 unique exercises that may be included in a PT treatment program, and the way a person performs each exercise in front of a camera can vary greatly. Additionally, in some examples, the patient may not even be performing the prescribed exercise, and may be engaging in a different activity. For effective development of an AI-based digital system to monitor PT activity, measurements collected when the patient is not performing the required exercise should disregarded, and focus should be on accurate measurement of the relevant metrics, such as repetition count and range of motion.

Many different issues that can arise while collecting measurements. For example, one potential issue is that the patient is performing an exercise in a way that is not well visible to the camera (e.g., with their back to the camera, too far from the camera, too close to the camera, partially obstructed by something in the scene, and/or partially obstructed by their own clothing). A second potential issue is that no person is detected in the scene (e.g., the patient leaves the area or the lighting conditions are poor). A third potential issue is that the patient performs the exercise in a plane that is harder to measure with a 2D camera (e.g., the person is standing facing the camera, in a frontal view, and moving their leg backward-standing sagittal to the camera would provide a better view of the movement). A fourth potential issue is that there is more than one person moving in the scene and it is difficult to identify the target patient. A fifth potential issue is that, for non-symmetric exercises, the patient performs the exercise with the incorrect body side or with the body side that is facing away from the camera, in a sagittal view. Another potential issue is that the patient is performing a different exercise altogether or not performing any exercise at all. Systems and methods are presented herein to use a deep learning system to guide the patient throughout the exercises and prevent or minimize the potential issues. If an issue does arise, the deep learning system can provide correction actions and/or instructions to the patient. Additionally, the deep learning system provides guidance and instructions that can enhance measurement accuracy for the system through corrective actions that can prevent and/or address issues. Providing real-time feedback improves treatment results by ensuring accurate performance of the exercise by the patient.

The guidance provided by the deep learning system can include pre-check feedback, in which guidance is provided before the patient begins the exercise. The guidance can include where to stand, how to stand, and how to start the exercise. The guidance can also include real-time feedback, in which guidance is provided while the patient is performing the exercise. Real-time feedback can be based on identified conditions that are not optimal for performing measurements of the exercise. In some examples, the real-time feedback can include guidance to correct the exercise, correct the side performing the exercise, correct the location relative to the camera, and/or to remove an obstruction between the patient and the camera.

For purposes of explanation, specific numbers, materials, and configurations are set forth in order to provide a thorough understanding of the illustrative implementations. However, it will be apparent to one skilled in the art that the present disclosure may be practiced without the specific details or/and that the present disclosure may be practiced with only some of the described aspects. In other instances, well known features are omitted or simplified in order not to obscure the illustrative implementations.

Further, references are made to the accompanying drawings that form a part hereof, and in which is shown, by way of illustration, embodiments that may be practiced. It is to be understood that other embodiments may be utilized, and structural or logical changes may be made without departing from the scope of the present disclosure. Therefore, the following detailed description is not to be taken in a limiting sense.

Various operations may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order from the described embodiment. Various additional operations may be performed or described operations may be omitted in additional embodiments.

For the purposes of the present disclosure, the phrase “A and/or B” or the phrase “A or B” means (A), (B), or (A and B). For the purposes of the present disclosure, the phrase “A, B, and/or C” or the phrase “A, B, or C” means (A), (B), (C), (A and B), (A and C), (B and C), or (A, B, and C). The term “between,” when used with reference to measurement ranges, is inclusive of the ends of the measurement ranges.

The description uses the phrases “in an embodiment” or “in embodiments,” which may each refer to one or more of the same or different embodiments. The terms “comprising,” “including,” “having,” and the like, as used with respect to embodiments of the present disclosure, are synonymous. The disclosure may use perspective-based descriptions such as “above,” “below,” “top,” “bottom,” and “side” to explain various features of the drawings, but these terms are simply for ease of discussion, and do not imply a desired or required orientation. The accompanying drawings are not necessarily drawn to scale. Unless otherwise specified, the use of the ordinal adjectives “first,” “second,” and “third,” etc., to describe a common object, merely indicates that different instances of like objects are being referred to and are not intended to imply that the objects so described must be in a given sequence, either temporally, spatially, in ranking or in any other manner.

In the following detailed description, various aspects of the illustrative implementations will be described using terms commonly employed by those skilled in the art to convey the substance of their work to others skilled in the art.

The terms “substantially,” “close,” “approximately,” “near,” and “about,” generally refer to being within +/−20% of a target value based on the input operand of a particular value as described herein or as known in the art. Similarly, terms indicating orientation of various elements, e.g., “coplanar,” “perpendicular,” “orthogonal,” “parallel,” or any other angle between the elements, generally refer to being within +/−5-20% of a target value based on the input operand of a particular value as described herein or as known in the art.

In addition, the terms “comprise,” “comprising,” “include,” “including,” “have,” “having” or any other variation thereof, are intended to cover a non-exclusive inclusion. For example, a method, process, device, or system that comprises a list of elements is not necessarily limited to only those elements but may include other elements not expressly listed or inherent to such method, process, device, or systems. Also, the term “or” refers to an inclusive “or” and not to an exclusive “or.”

The systems, methods, and devices of this disclosure each have several innovative aspects, no single one of which is solely responsible for all desirable attributes disclosed herein. Details of one or more implementations of the subject matter described in this specification are set forth in the description below and the accompanying drawings.

### Example DNN System

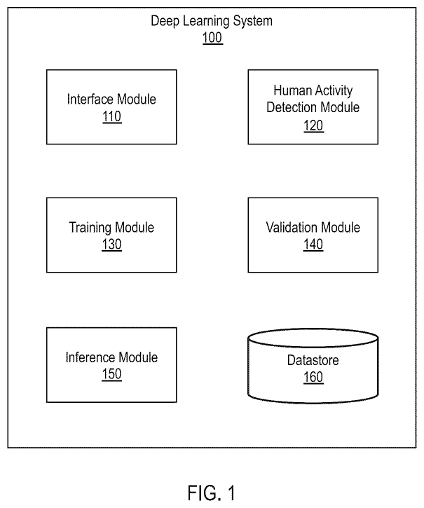

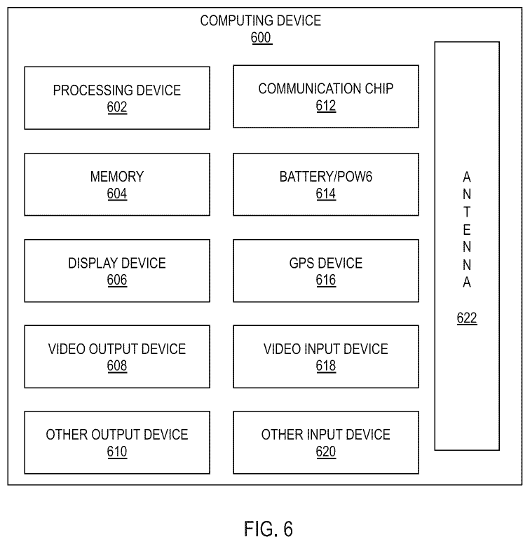

FIG. 1 is a block diagram of an example DNN system 100, in accordance with various embodiments. The DNN system 100 trains DNNs for various tasks, including physical therapy exercise analysis. The DNN system 100 includes an interface module 110, a physical therapy exercise analysis module 120, a training module 130, a validation module 140, an inference module 150, and a datastore 160. In other embodiments, alternative configurations, different or additional components may be included in the DNN system 100. Further, functionality attributed to a component of the DNN system 100 may be accomplished by a different component included in the DNN system 100 or a different system. The DNN system 100 or a component of the DNN system 100 (e.g., the training module 130 or inference module 150) may include the computing device 600 in FIG. 6.

The interface module 110 facilitates communications of the DNN system 100 with other systems. As an example, the interface module 110 supports the DNN system 100 to distribute trained DNNs to other systems, e.g., computing devices configured to apply DNNs to perform tasks. As another example, the interface module 110 establishes communications between the DNN system 100 with an external database to receive data that can be used to train DNNs or input into DNNs to perform tasks. In some embodiments, data received by the interface module 110 may have a data structure, such as a matrix. In some embodiments, data received by the interface module 110 may be an image, a series of images, and/or a video stream.

The physical therapy exercise analysis module 120 identifies an exerciser, determines that selected measurement conditions are met, and analyzes the exercises. The physical therapy exercise analysis module 120 performs physical therapy exercise analysis in real-time and provides real-time guidance to the exerciser. In general, the physical therapy exercise analysis module 120 includes multiple components which can perform functions such as pre-checks, providing pre-check guidance, live-checks, measuring and analyzing patient movement during exercises, and providing real-time guidance and feedback to the exerciser.

During training, the physical therapy exercise analysis module 120 can use a training data set including videos of an expert performing the exercises, with each video labeled with labels indicating which exercise is being performed, which side (if applicable), and how many repetitions are performed. In various examples, as described herein, the physical therapy exercise analysis module 120 includes one or more neural networks for processing input images and videos. In some examples, the physical therapy exercise analysis module 120 extracts skeleton sequences as described herein, and arranges the skeleton sequences in time as a 3D array. The physical therapy exercise analysis module 120 can arrange the joints from the skeleton sequence into multiple groups of joints, each representing a human body part that moves together when performing exercises. The movement of each group of joints over a selected time period (e.g., a few seconds) is represented in a matrix. In various implementations, the physical therapy exercise analysis module 120 includes a deep learning model for classifying the exercise based on the matrix data. In some examples, the deep learning model includes a transformer.

In various examples, as described herein, the physical therapy exercise analysis module 120 includes one or more neural networks for processing input videos. In some examples, the physical therapy exercise analysis module 120 includes one or more deep neural networks (DNN) for processing input images. The training module 130 trains DNNs using training datasets. In some embodiments, a training dataset for training a DNN may include one or more images and/or videos, each of which may be a training sample. In some examples, the training module 130 trains the physical therapy exercise analysis module 120. The training module 130 may receive real-world video data for processing with the physical therapy exercise analysis module 120 as described herein. In some embodiments, the training module 130 may input different data into different layers of the DNN. For every subsequent DNN layer, the input data may be less than the previous DNN layer. The training module 130 may adjust internal parameters of the DNN to minimize a difference between training data output and the input data processed by the physical therapy exercise analysis module 120.

In some embodiments, a part of the training dataset may be used to initially train the DNN, and the rest of the training dataset may be held back as a validation subset used by the validation module 140 to validate performance of a trained DNN. The portion of the training dataset not including the tuning subset and the validation subset may be used to train the DNN.

The training module 130 also determines hyperparameters for training the DNN. Hyperparameters are variables specifying the DNN training process. Hyperparameters are different from parameters inside the DNN (e.g., weights of filters). In some embodiments, hyperparameters include variables determining the architecture of the DNN, such as number of hidden layers, etc. Hyperparameters also include variables which determine how the DNN is trained, such as batch size, number of epochs, etc. A batch size defines the number of training samples to work through before updating the parameters of the DNN. The batch size is the same as or smaller than the number of samples in the training dataset. The training dataset can be divided into one or more batches. The number of epochs defines how many times the entire training dataset is passed forward and backwards through the entire network. The number of epochs defines the number of times that the deep learning algorithm works through the entire training dataset. One epoch means that each training sample in the training dataset has had an opportunity to update the parameters inside the DNN. An epoch may include one or more batches. The number of epochs may be 1, 10, 50, 100, or even larger.

The training module 130 defines the architecture of the DNN, e.g., based on some of the hyperparameters. The architecture of the DNN includes an input layer, an output layer, and a plurality of hidden layers. The input layer of an DNN may include tensors (e.g., a multidimensional array) specifying attributes of the input image, such as the height of the input image, the width of the input image, and the depth of the input image (e.g., the number of bits specifying the color of a pixel in the input image). The output layer includes labels of objects in the input layer. The hidden layers are layers between the input layer and output layer. The hidden layers include one or more convolutional layers and one or more other types of layers, such as pooling layers, fully connected layers, normalization layers, softmax or logistic layers, and so on. The convolutional layers of the DNN abstract the input image to a feature map that is represented by a tensor specifying the feature map height, the feature map width, and the feature map channels (e.g., red, green, blue images include 3 channels). A pooling layer is used to reduce the spatial volume of input image after convolution. It is used between 2 convolution layers. A fully connected layer involves weights, biases, and neurons. It connects neurons in one layer to neurons in another layer. It is used to classify images between different categories by training.

In the process of defining the architecture of the DNN, the training module 130 also adds an activation function to a hidden layer or the output layer. An activation function of a layer transforms the weighted sum of the input of the layer to an output of the layer. The activation function may be, for example, a rectified linear unit activation function, a tangent activation function, or other types of activation functions.

After the training module 130 defines the architecture of the DNN, the training module 130 inputs a training dataset into the DNN. The training dataset includes a plurality of training samples. An example of a training dataset includes a series of images of a video stream. Unlabeled, real-world video is input to the physical therapy exercise analysis module 120, and processed using the physical therapy exercise analysis module 120 parameters of the DNN to produce two different model-generated outputs: a first time-forward model-generated output and a second time-reversed model-generated output. In the backward pass, the training module 130 modifies the parameters inside the DNN (“internal parameters of the DNN”) to minimize the differences between the first model-generated output is and the second model generated output. The internal parameters include weights of filters in the convolutional layers of the DNN. In some embodiments, the training module 130 uses a cost function to minimize the differences.

The training module 130 may train the DNN for a predetermined number of epochs. The number of epochs is a hyperparameter that defines the number of times that the deep learning algorithm will work through the entire training dataset. One epoch means that each sample in the training dataset has had an opportunity to update internal parameters of the DNN. After the training module 130 finishes the predetermined number of epochs, the training module 130 may stop updating the parameters in the DNN. The DNN having the updated parameters is referred to as a trained DNN.

The validation module 140 verifies accuracy of trained DNNs. In some embodiments, the validation module 140 inputs samples in a validation dataset into a trained DNN and uses the outputs of the DNN to determine the model accuracy. In some embodiments, a validation dataset may be formed of some or all the samples in the training dataset. Additionally or alternatively, the validation dataset includes additional samples, other than those in the training sets. In some embodiments, the validation module 140 may determine an accuracy score measuring the precision, recall, or a combination of precision and recall of the DNN. The validation module 140 may use the following metrics to determine the accuracy score: Precision=TP/(TP+FP) and Recall=TP/(TP+FN), where precision may be how many the reference classification model correctly predicted (TP or true positives) out of the total it predicted (TP+FP or false positives), and recall may be how many the reference classification model correctly predicted (TP) out of the total number of objects that did have the property in question (TP+FN or false negatives). The F-score (F-score=2*PR/(P+R)) unifies precision and recall into a single measure.

The validation module 140 may compare the accuracy score with a threshold score. In an example where the validation module 140 determines that the accuracy score of the augmented model is lower than the threshold score, the validation module 140 instructs the training module 130 to re-train the DNN. In one embodiment, the training module 130 may iteratively re-train the DNN until the occurrence of a stopping condition, such as the accuracy measurement indication that the DNN may be sufficiently accurate, or a number of training rounds having taken place.

The inference module 150 applies the trained or validated DNN to perform tasks. The inference module 150 may run inference processes of a trained or validated DNN. In some examples, inference makes use of the forward pass to produce model-generated output for unlabeled real-world data. For instance, the inference module 150 may input real-world data into the DNN and receive an output of the DNN. The output of the DNN may provide a solution to the task for which the DNN is trained for.

The inference module 150 may aggregate the outputs of the DNN to generate a final result of the inference process. In some embodiments, the inference module 150 may distribute the DNN to other systems, e.g., computing devices in communication with the DNN system 100, for the other systems to apply the DNN to perform the tasks. The distribution of the DNN may be done through the interface module 110. In some embodiments, the DNN system 100 may be implemented in a server, such as a cloud server, an edge service, and so on. The computing devices may be connected to the DNN system 100 through a network. Examples of the computing devices include edge devices.

The datastore 160 stores data received, generated, used, or otherwise associated with the DNN system 100. For example, the datastore 160 stores video processed by the physical therapy exercise analysis module 120 or used by the training module 130, validation module 140, and the inference module 150. The datastore 160 may also store other data generated by the training module 130 and validation module 140, such as the hyperparameters for training DNNs, internal parameters of trained DNNs (e.g., values of tunable parameters of activation functions, such as Fractional Adaptive Linear Units (FALUs)), etc. In the embodiment of FIG. 1, the datastore 160 is a component of the DNN system 100. In other embodiments, the datastore 160 may be external to the DNN system 100 and communicate with the DNN system 100 through a network.

### Example Exercise Analysis System

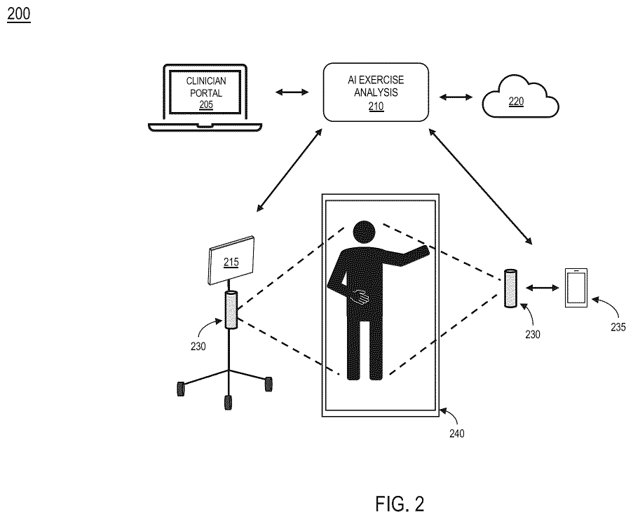

FIG. 2 illustrates an example overview of an exercise analysis system 200, in accordance with various embodiments. In particular, various parts of an exercise analysis system 200 are illustrated in FIG. 2. The exercise analysis system 200 includes an artificial intelligence (AI)-based exercise analysis module 210, a clinician portal 205, a patient application 215, a cloud 220, and a camera 230. In some examples, the camera 230 is a mobile device 235 camera. A user can set up the system such that the camera 230 has a field of view 240 focused on the user. Recordings from the camera 230 can be shared with the AI-based exercise analysis module 210, which can evaluate the recordings in real time and provide feedback and/or guidance to the user.

In some implementations, the exercise analysis system 200 can be used by physical therapists as an AI-guided physical therapy system. A mobile device application, such as on a tablet or phone, can provide the patient application 215 and serve as the user interface to the patient. The patient application 215 can communicate with the patient, the camera 230, the AI-based exercise analysis module 210, and the cloud 220. The camera 230 is positioned in front of the patient and streams video in real time to the mobile device (e.g., mobile device 235) and/or to the AI-based exercise analysis module 210. At the AI-based exercise analysis module 210, the patient movements and environment are analyzed, and guidance is generated to provide to the patient. In some examples, the patient can interact with the AI-based exercise analysis module 210 via the patient application 215. For instance, the patient application 215 may guide the patient visually and/or audibly. The AI-based exercise analysis module 210 receives the video stream from the camera 230, analyzes the video, extracts data from the video, and transmits instructions to the patient in real-time, including pre-checks and/or live-checks.

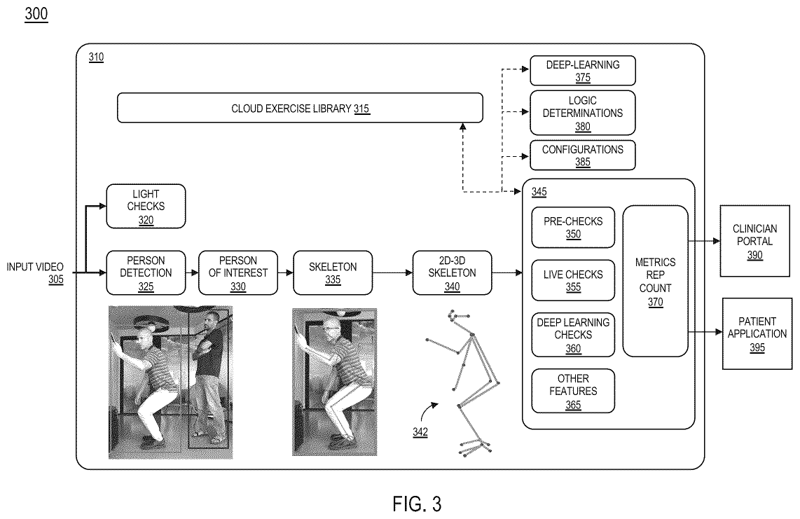

For physical therapists, clinicians, coaches, etc., the clinician portal 205 can present real-time and/or end-of-session measurement results and corresponding video or image frames. In particular, the clinician portal 205 receives the results from the AI-based exercise analysis module 210. FIG. 3 illustrates an example of a pipeline for implementation of the AI-based exercise analysis module 210.

In particular, as shown in FIG. 3, an input video 305 is received at a human activity analysis system 310. The human activity analysis system 310 includes an exercise library 315, a light check module 320, a person detection module 325, a person-of-interest identification module 330, a skeleton identification module 335, a 2D-3D skeleton generation module 340, an analysis module 345, a deep-learning module 375, a logic determinations module 380, and a configurations module 385. The human activity analysis system 310 generates outputs to a clinician portal 390 and to a patient application 395.

In various examples, the exercise library 315 includes a set of exercises that a user may be performing. The set of exercises can include PT exercises. The human activity analysis system 310 receives an input video 305, and inputs the video to a light checks module 320. The light checks module 320 determines whether the lighting in the video is sufficient for detection (and analysis) of the person in the video. If the lighting is insufficient, the human activity analysis system 310 can output an alert to the patient application 395 to adjust lighting in the area in which the patient is performing (and recording) the exercises. The patient can then turn on lights, open curtains, or otherwise adjust the lighting until the light check alert from the light checks module 320 of the human activity analysis system 310 is no longer activated.

The human activity analysis system 310 receives the input video 305 at a person detection module 325, where any people present in the video are detected. At the person-of-interest module 330, the person performing the exercises is identified. Thus, if there are any bystanders or others in the room with the person performing the exercises, the system will remove the other people from its subsequent analysis. At the skeleton module 335, joints of the person performing the exercises are identified, and lines between the joints are generated to approximate a simplified skeleton. At the 2D-3D skeleton module 340, a simplified skeleton of the person performing the exercises is generated, as illustrated in the skeleton line-drawing 342 of FIG. 3. The 2D-3D skeleton module 340 saves the movement of the skeleton line-drawing 342 over time as skeleton sequences.

The skeleton sequences are output from the 2D-3D skeleton module 340 to an analysis module 345. The analysis module 345 performs pre-checks at the pre-checks module 350. The pre-checks can include checking where the exerciser is standing within the frame, how the exerciser is standing, lighting checks, and checks for obstructions. Pre-checks are discussed in greater detail below. The analysis module 345 performs live-checks at the live-checks module 355. The live-checks can include checks while the patient is performing the exercise, and can include, for example, guidance to correct the exercise, correct the side performing the exercise, correct the location relative to the camera, correct lighting, and/or to remove an obstruction between the patient and the camera. In some examples, the deep learning checks module 360 performs AI-based exercise analysis, and can communicate with the live-checks module 355 to provide guidance and feedback on exercise movements. In some examples, the deep learning checks module 360 analyzes the skeleton sequences and identifies any anomalies. The analysis module 345 include a metric rep count module 370, which can count the repetitions as the user performs the exercises. In various examples, the analysis module 345 can access the exercise library 315, a deep-learning system 375, logic determinations module 380, and configurations module 385. The analysis module 345 outputs its analysis to a clinician portal 390 and/or to a patient application 395.

In some examples, the analysis module 345 analyzes video clips that are around a few seconds long. For instance, the analysis module 345 may analyze video clips that are between about two seconds long and about four seconds long. In one example, the analysis module 345 analyzes video clips that are about three seconds long. The video clips can be overlapping, such that, in one example, a first video clip includes image frames from time zero seconds to time three seconds, a second video clip includes image frames from time one second to time four seconds, and a third video clip includes image frames from time two seconds to time five seconds.

The clinician portal 390 can display a video of the patient performing the exercise and a graph illustrating movement over time. In some examples, the clinician portal 390 can display measurement results transmitted to the clinician during and/or after completion of the exercise. The patient application portal 395 can display a video of the real-time video stream transmitted as the input video to the exercise analysis system 310. Instructions and/or guidance can be superimposed on the video image, where instructions and/or guidance can include feedback to the exerciser. The instructions and/or guidance are described in greater detail herein.

In various implementations, the human activity analysis system 310 is developed and fine-tuned to balance user experience, accuracy of exercise performance, and precision of physical therapy metrics. In some examples, the analysis module 345 determines when guiding conditions provided to the exerciser are met, and then determines measurements of various parameters. Thus, for instance, when exercise performance accuracy exceeds a selected threshold, the human activity analysis system 310 determines various exercise movement measurements. The measurements can include, for example, range of motion, repetition count, speed of each repetition, distance moved for one or more joints (average distance and/or distance for each repetition and/or change in distance moved with subsequent repetitions), and so on.

In some examples, the human activity analysis system 310 outputs a video of a person performed the selected exercise to the patient application 395. The cloud exercise library 315 can include a video dataset of people performing the supported exercises. The video dataset can include examples of people performing each exercise accurately, including good start positions, and accurate movement, and the video dataset can include examples of people performing exercises poorly, including inaccurate start positions and/or inaccurate movement. The video dataset can be used to tune checks of the exerciser for best metric accuracy of the parameters measured.

### Example Exercise Analysis Method

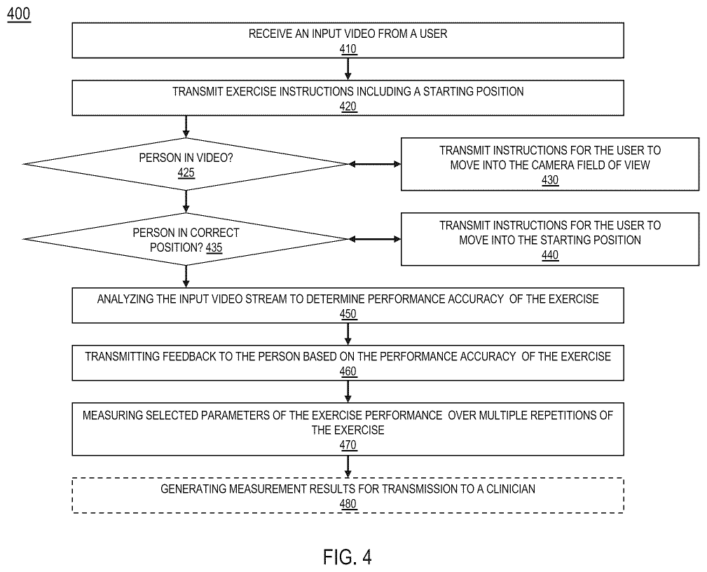

FIG. 4 is a flowchart showing a method 400 of exercise analysis, in accordance with various embodiments. The method 400 may be performed by the deep learning system 100 in FIG. 1, the exercise analysis system 200 in FIG. 2, and/or the. Although the method 400 is described with reference to the flowchart illustrated in FIG. 4, many other methods for exercise analysis may alternatively be used. For example, the order of execution of the elements of FIG. 4 may be changed. As another example, some of the elements may be changed, eliminated, or combined.

In various implementations, the method 400 can be used for interaction between an AI system (the exercise analysis system) and a user (the exerciser) to increase measurement accuracy of exercises. The exercise analysis system can provide guidance to the user to prevent measurement issues. For example, the exercise analysis system can suggest corrective actions when issues are identified. In some examples, the issues can include visual issues for video stream, such as position of the patient within the field of view (e.g., too far, too close, not centered, etc.), lighting issues, and obstructions between the patient and the camera. In some examples, the issues can include inaccuracies in exercise performance, such as incorrect exercise, incorrect movement of one or more body parts, incorrect side of body performing the movement, etc. In various examples, the exercise analysis system can perform pre-checks, completed before the user begins the exercise, as well as live-checks, completed while the user is performing the exercise.

Referring to the method 400 of FIG. 4, at 410, an input video is received from a user at an exercise analysis system. The input video can be a real-time video stream from a user device, such as a user tablet, phone, laptop, or other mobile device. At 420, the exercise analysis system transmits exercise instructions to the user. The exercise instructions can include instructions to the patient on where to stand and how to start the exercise. At 425, it is determined whether a person is in the video stream. If no person is found in the images of the video stream, at 430, instructions are transmitted to the user to move into the camera field of view. In some examples, if multiple people are found in the video stream, instructions can be transmitted to the user to stand in a selected position and for other people to move out of a selected area in the video stream. In some examples, when multiple people are in the field of view of the camera, once the person of interest is identified and passed a pre-check, the exercise analysis system “locks” on the person of interest to ensure best accuracy during performance of the exercise.

If at 425, a person is found in the video stream, at 435, it is determined whether the person is standing (or otherwise positioned) in the correct position for the exercise. In some examples, it is determined whether the person is in the correct position within images of the input video. For instance, if can be determined whether the person is too far from the camera and/or whether the person is too close to the camera. Similarly, it can be determined whether the person is off to one side in the images captured in the input video. The exercise analysis system can provide feedback to the person to move closer to the camera, further from the camera, to the right, to the left, etc. In some examples, the exercise analysis system guides the person to a best location for the exercise analysis system to analyze the exercises and provide helpful results. In various examples, the best location can vary depending on the exercise and the depending on the height of the person. In some examples, the exercise analysis system records patient height at first login of the patient, and scales its analysis based on the patient's height. In some examples, the best location can be calibrated based on a test set and/or based on benchmark results.

In some examples, determining whether the person is in the correct position includes determining whether the person is in the correct starting posture and/or position for the selected exercise. Starting postures and/or positions can include standing, seated, quadruped, supine, and so on. Additionally starting postures and/or positions can include various limb positions. Accurate starting position is useful for accurate execution of the exercise analysis system. Providing feedback before the person begins the exercise increases exercise performance accuracy and hence increases measurement accuracy of the exercise analysis system.

In some examples, determining whether the person is in the correct position includes determining whether the person is starting in the correct view and/or plane relative to the camera (i.e., frontal, sagittal left/right, posterior or transverse).

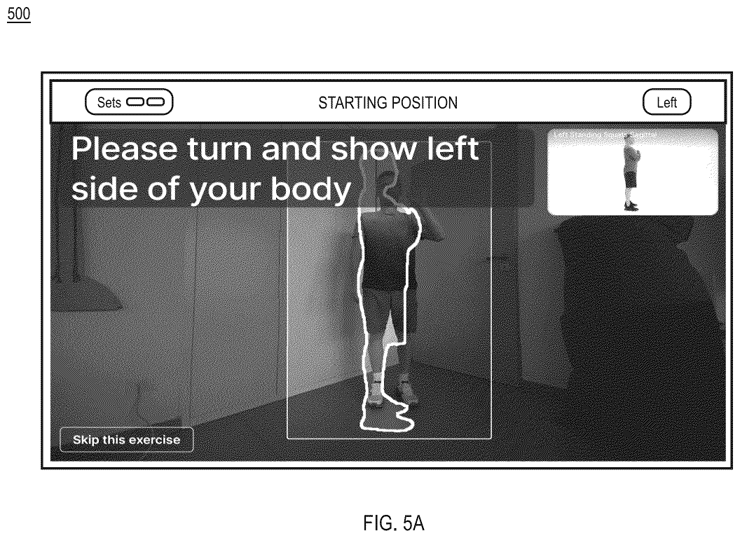

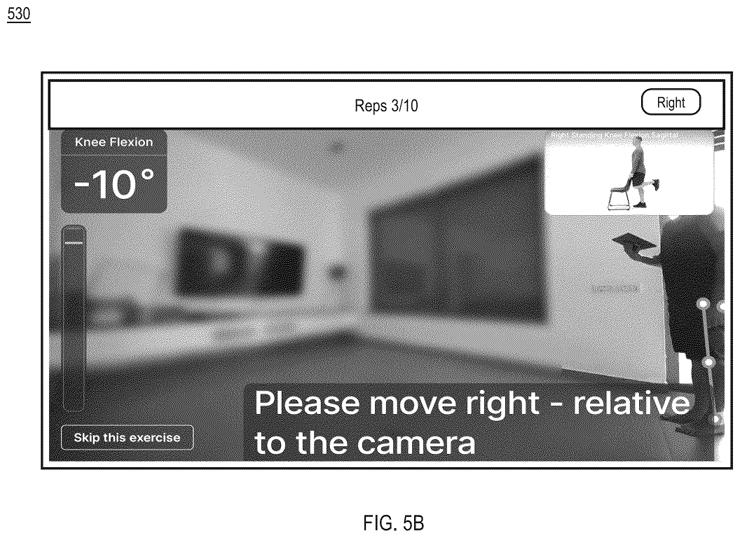

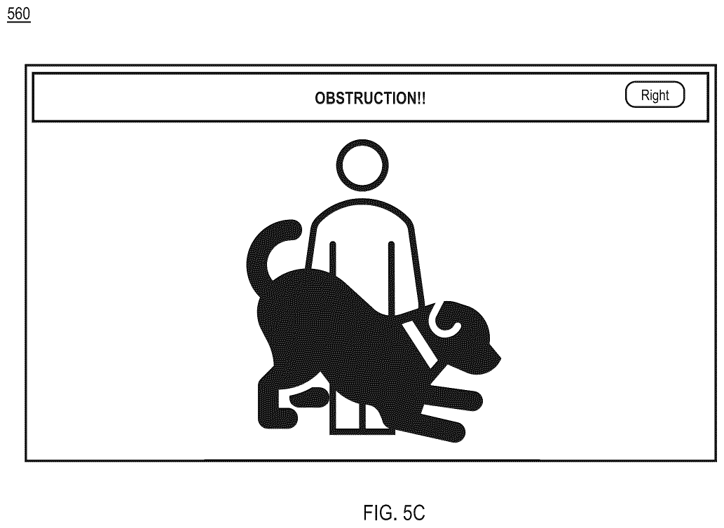

In various examples, if the person is not in the correct position, at 440, instructions are transmitted to the user to move to the starting position. FIG. 5A shows an example image from a video stream illustrating an outline highlighting the user starting position, in accordance with various embodiments. As shown in FIG. 5A, the outline is superimposed on the video stream. In FIG. 5A, the outline illustrates a left side of the body, and guidance is provided to the person to turn and show the left side of their body. In some examples, the person can position themselves such that they appear in the outline superimposed on the screen.

Additionally, as shown in FIG. 5A, a video is displayed in the top right of the image showing a person in the correct starting position. Ensuring the person starts the exercise from the correct starting position ensures that the patient plane shown to the camera (and displayed in the video stream) is the best plane for recording accurate measurements of the selected exercise. For example, when performing flexion/extension movements, the measurement is more accurate when seen in a sagittal view. In abduction/adduction movements, the measurement is more accurate in frontal view. For non-symmetric exercises, when the exercise is performed on the left body side, the measurement is more accurate when the left side is closer to the camera. Similarly, for non-symmetric exercises, when the exercise is performed on the right body side, the measurement is more accurate when the right side is closer to the camera. This check is performed for both 2D cameras and 3D cameras.

Another check that can be completed before the person begins performing the exercise is a lighting check. The lighting of the images in the video stream is checked for various issues, such as to ensure that the video is not too dark, not too bright, has no contrast issues such as high-dynamic range, and so on. Another check that may be completed is a human-equipment interaction test. In particular, if equipment is required to perform the exercise, an equipment check is performed to ensure the equipment is acceptable for the exercise. For example, if the exercise starts when the patient is standing on a box, the equipment check makes sure the person has an acceptable box and that the person is still in a good position relative to the camera when standing on the box. In another example, if a patient is lying on a physio ball for an exercise, the size and inflation of the physio ball can be checked.

Another check that can be completed before the person begins performing the exercise is to check for any obstructions between the person and the camera. For instance, exercise equipment may have been placed between the person and the camera, obstructing a view of the person. In another example, the person's clothing can be an obstruction. For instance, baggy clothes or a dress can obscure the movement.

When the pre-checks are completed and the person begins performing the exercise, additional checks are repeated during the exercise. The additional checks during the exercise serve to identify sub-optimal conditions for measurements by the exercise analysis system.

Referring to FIG. 4, at 450, the input video stream is analyzed to determine performance accuracy of the exercise. Examples of exercise input that can be checked during the exercise include determining whether the patient is performing the correct exercise, determining whether the patient is exercising the correct side (for non-symmetric exercises), and determining whether the patient is still positioned in the correct location relative to the camera. In some examples, an exercise may instruct a patient to move from one side of the room to another side of the room, and the patient may move out of the field of view of the camera. At 460, feedback can be transmitted to the person based on the checks and the identified performance accuracy of the exercise. Similar to the pre-checks, the input video stream can also be analyzed to identify any obstructions by objects, pets, equipment, clothing, etc., the lighting conditions can be checked, and the continued identification of the person-of-interest can be checked. Additional checks may include validating that the movement is performed in the expected plane (e.g., walking in a straight line and maintaining a fixed a distance from the camera throughout the exercise).

FIG. 5B shows an example image 530 from a video stream illustrating an example in which the person is no longer positioned in the correct location relative to the camera, in accordance with various embodiments. As shown in FIG. 5B, the exerciser is off to the left side of the image. Feedback is provided to the exerciser to move to the right relative to the camera, so that the exerciser will be back in the field of view of the camera.

FIG. 5C is a schematic showing an example image 560 from a video stream illustrating an example in which the view of the person is obstructed, in accordance with various embodiments. In particular, as show in FIG. 5C, a dog is sitting between the person and the camera, blocking the view of the person.

At 470, selected parameters of the exercise performance are measured over multiple repetitions of the exercises. The measurements can include parameters related to, for example, range of motion, repetition count, speed of each repetition, distance moved for one or more joints (average distance and/or distance for each repetition and/or change in distance moved with subsequent repetitions), and so on.

At 480, measurement results are generated for transmission to a clinician. In particular, if the person performing the exercises is working with a physical therapist, coach, personal trainer, clinician, etc., the measurement results for each exercise can be transmitted to that person for review and to provide further guidance to the exerciser. The measurement results can be used to correct accuracy of performance, to select future exercises, and/or to determine a next exercise protocol.

### Example Computing Device

FIG. 6 is a block diagram of an example computing device 600, in accordance with various embodiments. In some embodiments, the computing device 600 may be used for at least part of the deep learning system 100 in FIG. 1. A number of components are illustrated in FIG. 6 as included in the computing device 600, but any one or more of these components may be omitted or duplicated, as suitable for the application. In some embodiments, some or all of the components included in the computing device 600 may be attached to one or more motherboards. In some embodiments, some or all of these components are fabricated onto a single system on a chip (SoC) die. Additionally, in various embodiments, the computing device 600 may not include one or more of the components illustrated in FIG. 6, but the computing device 600 may include interface circuitry for coupling to the one or more components. For example, the computing device 600 may not include a display device 606, but may include display device interface circuitry (e.g., a connector and driver circuitry) to which a display device 606 may be coupled. In another set of examples, the computing device 600 may not include a video input device 618 or a video output device 608, but may include video input or output device interface circuitry (e.g., connectors and supporting circuitry) to which a video input device 618 or video output device 608 may be coupled.

The computing device 600 may include a processing device 602 (e.g., one or more processing devices). The processing device 602 processes electronic data from registers and/or memory to transform that electronic data into other electronic data that may be stored in registers and/or memory. The computing device 600 may include a memory 604, which may itself include one or more memory devices such as volatile memory (e.g., DRAM), nonvolatile memory (e.g., read-only memory (ROM)), high bandwidth memory (HBM), flash memory, solid state memory, and/or a hard drive. In some embodiments, the memory 604 may include memory that shares a die with the processing device 602. In some embodiments, the memory 604 includes one or more non-transitory computer-readable media storing instructions executable for occupancy mapping or collision detection, e.g., the method 400 described above in conjunction with FIG. 4 or some operations performed by the DNN system 100 in FIG. 1, the system 200 of FIG. 2, and/or the system 300 of FIG. 3. The instructions stored in the one or more non-transitory computer-readable media may be executed by the processing device 602.

In some embodiments, the computing device 600 may include a communication chip 612 (e.g., one or more communication chips). For example, the communication chip 612 may be configured for managing wireless communications for the transfer of data to and from the computing device 600. The term “wireless” and its derivatives may be used to describe circuits, devices, systems, methods, techniques, communications channels, etc., that may communicate data using modulated electromagnetic radiation through a nonsolid medium. The term does not imply that the associated devices do not contain any wires, although in some embodiments they might not.

The communication chip 612 may implement any of a number of wireless standards or protocols, including but not limited to Institute for Electrical and Electronic Engineers (IEEE) standards including Wi-Fi (IEEE 802.10 family), IEEE 802.16 standards (e.g., IEEE 802.16-2005 Amendment), Long-Term Evolution (LTE) project along with any amendments, updates, and/or revisions (e.g., advanced LTE project, ultramobile broadband (UMB) project (also referred to as “3GPP2”), etc.). IEEE 802.16 compatible Broadband Wireless Access (BWA) networks are generally referred to as WiMAX networks, an acronym that stands for worldwide interoperability for microwave access, which is a certification mark for products that pass conformity and interoperability tests for the IEEE 802.16 standards. The communication chip 612 may operate in accordance with a Global System for Mobile Communication (GSM), General Packet Radio Service (GPRS), Universal Mobile Telecommunications System (UMTS), High Speed Packet Access (HSPA), Evolved HSPA (E-HSPA), or LTE network. The communication chip 612 may operate in accordance with Enhanced Data for GSM Evolution (EDGE), GSM EDGE Radio Access Network (GERAN), Universal Terrestrial Radio Access Network (UTRAN), or Evolved UTRAN (E-UTRAN). The communication chip 612 may operate in accordance with code-division multiple access (CDMA), Time Division Multiple Access (TDMA), Digital Enhanced Cordless Telecommunications (DECT), Evolution-Data Optimized (EV-DO), and derivatives thereof, as well as any other wireless protocols that are designated as 3G, 4G, 5G, and beyond. The communication chip 612 may operate in accordance with other wireless protocols in other embodiments. The computing device 600 may include an antenna 622 to facilitate wireless communications and/or to receive other wireless communications (such as AM or FM radio transmissions).

In some embodiments, the communication chip 612 may manage wired communications, such as electrical, optical, or any other suitable communication protocols (e.g., the Ethernet). As noted above, the communication chip 612 may include multiple communication chips. For instance, a first communication chip 612 may be dedicated to shorter-range wireless communications such as Wi-Fi or Bluetooth, and a second communication chip 612 may be dedicated to longer-range wireless communications such as global positioning system (GPS), EDGE, GPRS, CDMA, WiMAX, LTE, EV-DO, or others. In some embodiments, a first communication chip 612 may be dedicated to wireless communications, and a second communication chip 612 may be dedicated to wired communications.

The computing device 600 may include battery/power circuitry 614. The battery/power circuitry 614 may include one or more energy storage devices (e.g., batteries or capacitors) and/or circuitry for coupling components of the computing device 600 to an energy source separate from the computing device 600 (e.g., AC line power).

The computing device 600 may include a display device 606 (or corresponding interface circuitry, as discussed above). The display device 606 may include any visual indicators, such as a heads-up display, a computer monitor, a projector, a touchscreen display, a liquid crystal display (LCD), a light-emitting diode display, or a flat panel display, for example.

The computing device 600 may include a video output device 608 (or corresponding interface circuitry, as discussed above). The video output device 608 may include any device that generates an audible indicator, such as speakers, headsets, or earbuds, for example.

The computing device 600 may include a video input device 618 (or corresponding interface circuitry, as discussed above). The video input device 618 may include any device that generates a signal representative of a sound, such as microphones, microphone arrays, or digital instruments (e.g., instruments having a musical instrument digital interface (MIDI) output).

The computing device 600 may include a GPS device 616 (or corresponding interface circuitry, as discussed above). The GPS device 616 may be in communication with a satellite-based system and may receive a location of the computing device 600, as known in the art.

The computing device 600 may include another output device 610 (or corresponding interface circuitry, as discussed above). Examples of the other output device 610 may include a video codec, a video codec, a printer, a wired or wireless transmitter for providing information to other devices, or an additional storage device.

The computing device 600 may include another input device 620 (or corresponding interface circuitry, as discussed above). Examples of the other input device 620 may include an accelerometer, a gyroscope, a compass, an image capture device, a keyboard, a cursor control device such as a mouse, a stylus, a touchpad, a bar code reader, a Quick Response (QR) code reader, any sensor, or a radio frequency identification (RFID) reader.

The computing device 600 may have any desired form factor, such as a handheld or mobile computer system (e.g., a cell phone, a smart phone, a mobile internet device, a music player, a tablet computer, a laptop computer, a netbook computer, an ultrabook computer, a personal digital assistant (PDA), an ultramobile personal computer, etc.), a desktop computer system, a server or other networked computing component, a printer, a scanner, a monitor, a set-top box, an entertainment control unit, a vehicle control unit, a digital camera, a digital video recorder, or a wearable computer system. In some embodiments, the computing device 600 may be any other electronic device that processes data.

### Selected Examples

The following paragraphs provide various examples of the embodiments disclosed herein.
Example 1 provides a computer-implemented method, including receiving a real-time input video stream; transmitting exercise instructions for a person to perform a selected exercise; determining the person is present in the real-time input video stream; analyzing a first video segment of the real-time input video stream, at a neural network, to determine performance of the selected exercise; transmitting feedback to the person based on the performance; measuring selected parameters of the performance of the selected exercise over a plurality of repetitions of the selected exercise; and generating measurement results.
Example 2 provides the computer-implemented method according to example 1, where transmitting exercise instructions includes transmitting an outline of a starting position for the selected exercise, and where the outline can be superimposed on the real-time input video stream.
Example 3 provides the computer-implemented method according to examples 1-2, further including identifying a present position of the person in the real-time input video stream and transmitting position instructions for the person to move to a best position based on selected exercise.
Example 4 provides the computer-implemented method according to example 1-3, further including identifying an obstruction in the real-time input video stream blocking a view of the person, and transmitting instructions to move the obstruction.
Example 5 provides the computer-implemented method according to example 1-4, where analyzing the first video segment of the real-time input video stream includes identifying a side of the person performing the selected exercise, where the side is one of a right side, a left side, and both sides.
Example 6 provides the computer-implemented method according to example 5, further including determining the person is performing the selected exercise on a wrong side and transmitting feedback to switch sides.
Example 7 provides the computer-implemented method according to example 1-6, where the measurement results includes a total number of repetitions of the selected exercise performed.
Example 8 provides the computer-implemented method according to example 1-7, where transmitting exercise instructions includes transmitting a video of a person performing the selected exercise.
Example 9 provides one or more non-transitory computer-readable media storing instructions executable to perform operations, the operations including receiving a real-time input video stream; transmitting exercise instructions for a person to perform a selected exercise; determining the person is present in the real-time input video stream; analyzing a first video segment of the real-time input video stream, at a neural network, to determine performance of the selected exercise; transmitting feedback to the person based on the performance; measuring selected parameters of the performance of the selected exercise over a plurality of repetitions of the selected exercise; and generating measurement results.
Example 10 provides the one or more non-transitory computer-readable media according to example 9, where transmitting exercise instructions includes transmitting an outline of a starting position for the selected exercise, and where the outline can be superimposed on the real-time input video stream.
Example 11 provides the one or more non-transitory computer-readable media according to example 9-10, further including identifying a present position of the person in the real-time input video stream and transmitting position instructions for the person to move to a best position based on selected exercise.
Example 12 provides the one or more non-transitory computer-readable media according to example 9-11, further including identifying an obstruction in the real-time input video stream blocking a view of the person, and transmitting instructions to move the obstruction.
Example 13 provides the one or more non-transitory computer-readable media according to example 9-12, where analyzing the first video segment of the real-time input video stream includes identifying a side of the person performing the selected exercise, where the side is one of a right side, a left side, and both sides.
Example 14 provides the one or more non-transitory computer-readable media according to example 13, further including determining the person is performing the selected exercise on a wrong side and transmitting feedback to switch sides.
Example 15 provides the one or more non-transitory computer-readable media according to example 9-14, where the measurement results includes a total number of repetitions of the selected exercise performed.
Example 16 provides the one or more non-transitory computer-readable media according to example 9-15, where transmitting exercise instructions includes transmitting a video of a person performing the selected exercise.
Example 17 provides an apparatus, including a computer processor for executing computer program instructions; and a non-transitory computer-readable memory storing computer program instructions executable by the computer processor to perform operations including receiving a real-time input video stream; transmitting exercise instructions for a person to perform a selected exercise; determining the person is present in the real-time input video stream; analyzing a first video segment of the real-time input video stream, at a neural network, to determine performance of the selected exercise; transmitting feedback to the person based on the performance; measuring selected parameters of the performance of the selected exercise over a plurality of repetitions of the selected exercise; and generating measurement results.
Example 18 provides the apparatus according to example 17, where transmitting exercise instructions includes transmitting an outline of a starting position for the selected exercise, and where the outline can be superimposed on the real-time input video stream.
Example 19 provides the apparatus according to example 17-18, further including identifying a present position of the person in the real-time input video stream and transmitting position instructions for the person to move to a best position based on selected exercise.
Example 20 provides the apparatus according to example 17-19, where analyzing the first video segment of the real-time input video stream includes identifying a side of the person performing the selected exercise, where the side is one of a right side, a left side, and both sides.

The above description of illustrated implementations of the disclosure, including what is described in the Abstract, is not intended to be exhaustive or to limit the disclosure to the precise forms disclosed. While specific implementations of, and examples for, the disclosure are described herein for illustrative purposes, various equivalent modifications are possible within the scope of the disclosure, as those skilled in the relevant art will recognize. These modifications may be made to the disclosure in light of the above detailed description.

1. A computer-implemented method, comprising:
receiving a real-time input video stream;
transmitting exercise instructions for a person to perform a selected exercise;
determining the person is present in the real-time input video stream;
analyzing a first video segment of the real-time input video stream, at a neural network, to determine performance of the selected exercise;
transmitting feedback to the person based on the performance;
measuring selected parameters of the performance of the selected exercise over a plurality of repetitions of the selected exercise; and
generating measurement results.

2. The computer-implemented method according to claim 1, wherein transmitting exercise instructions includes transmitting an outline of a starting position for the selected exercise, and wherein the outline can be superimposed on the real-time input video stream.

3. The computer-implemented method according to claim 1, further comprising identifying a present position of the person in the real-time input video stream and transmitting position instructions for the person to move to a target position based on the selected exercise.

4. The computer-implemented method according to claim 1, further comprising identifying an obstruction in the real-time input video stream blocking a view of the person, and transmitting instructions to move the obstruction.

5. The computer-implemented method according to claim 1, wherein analyzing the first video segment of the real-time input video stream includes identifying a side of the person performing the selected exercise, wherein the side is one of a right side, a left side, and both sides.

6. The computer-implemented method according to claim 5, further comprising determining the person is performing the selected exercise on a wrong side and transmitting feedback to switch sides.

7. The computer-implemented method according to claim 1, wherein the measurement results includes a total number of repetitions of the selected exercise performed.

8. The computer-implemented method according to claim 1, wherein transmitting exercise instructions includes transmitting a video of a person performing the selected exercise.

9. One or more non-transitory computer-readable media storing instructions executable to perform operations, the operations comprising:
receiving a real-time input video stream;
transmitting exercise instructions for a person to perform a selected exercise;
determining the person is present in the real-time input video stream;
analyzing a first video segment of the real-time input video stream, at a neural network, to determine performance of the selected exercise;
transmitting feedback to the person based on the performance;
measuring selected parameters of the performance of the selected exercise over a plurality of repetitions of the selected exercise; and
generating measurement results.

10. The one or more non-transitory computer-readable media according to claim 9, wherein transmitting exercise instructions includes transmitting an outline of a starting position for the selected exercise, and wherein the outline can be superimposed on the real-time input video stream.

11. The one or more non-transitory computer-readable media according to claim 9, further comprising identifying a present position of the person in the real-time input video stream and transmitting position instructions for the person to move to a target position based on the selected exercise.

12. The one or more non-transitory computer-readable media according to claim 9, further comprising identifying an obstruction in the real-time input video stream blocking a view of the person, and transmitting instructions to move the obstruction.

13. The one or more non-transitory computer-readable media according to claim 9, wherein analyzing the first video segment of the real-time input video stream includes identifying a side of the person performing the selected exercise, wherein the side is one of a right side, a left side, and both sides.

14. The one or more non-transitory computer-readable media according to claim 13, further comprising determining the person is performing the selected exercise on a wrong side and transmitting feedback to switch sides.

15. The one or more non-transitory computer-readable media according to claim 9, wherein the measurement results includes a total number of repetitions of the selected exercise performed.

16. The one or more non-transitory computer-readable media according to claim 9, wherein transmitting exercise instructions includes transmitting a video of a person performing the selected exercise.

17. An apparatus, comprising:
a computer processor for executing computer program instructions; and
a non-transitory computer-readable memory storing computer program instructions executable by the computer processor to perform operations comprising:
receiving a real-time input video stream;
transmitting exercise instructions for a person to perform a selected exercise;
determining the person is present in the real-time input video stream;
analyzing a first video segment of the real-time input video stream, at a neural network, to determine performance of the selected exercise;
transmitting feedback to the person based on the performance;
measuring selected parameters of the performance of the selected exercise over a plurality of repetitions of the selected exercise; and
generating measurement results.

18. The apparatus according to claim 17, wherein transmitting exercise instructions includes transmitting an outline of a starting position for the selected exercise, and wherein the outline can be superimposed on the real-time input video stream.

19. The apparatus according to claim 17, further comprising identifying a present position of the person in the real-time input video stream and transmitting position instructions for the person to move to a target position based on the selected exercise.

20. The apparatus according to claim 17, wherein analyzing the first video segment of the real-time input video stream includes identifying a side of the person performing the selected exercise, wherein the side is one of a right side, a left side, and both sides.
